(19) United States
(12) Plant Patent Application Publication
(10) Pub. No.: US 2012/0255079 P1
(43) Pub. Date: Oct. 4, 2012

(54) Alstroemeria plant named 'Zalsamida'

(50) ;

(75) Inventor: Henricus Cornelius Maria Jacobs, Rijsenhout (NL)

(73) Assignee: van Zanten Plants b.v., Hillegom (NL)

(21) Appl. No.: 13/385,872

(22) Filed: Mar. 10, 2012

(30) Foreign Application Priority Data
Mar. 30, 2011 (EP) 2011/0782

(51) Int. Cl. A01H 5/00

(52) U.S. Cl. PLT/309

(57) ABSTRACT

A new and distinct cultivar of Alstroemeria plant named ‘Zalsamida’, characterized by its erect and strong flowering stems; vigorous growth habit; purple and white-colored flowers; and excellent postproduction longevity.

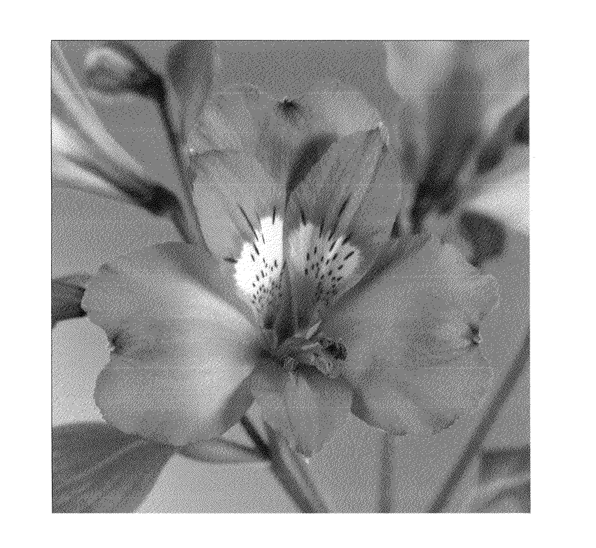

### BOTANICAL DESIGNATION

Alstroemeria hybrida

### CULTIVAR DENOMINATION

‘ZALSAMIDA’

### BACKGROUND OF THE INVENTION

The present invention relates to a new and distinct cultivar of Alstroemeria plant, botanically known as Alstroemeria hybrida, commercially used as a cut flower Alstroemeria, and hereinafter referred to by the name ‘Zalsamida’.

The new Alstroemeria plant is a product of a planned breeding program conducted by the Inventor in Rijsenhout, The Netherlands. The objective of the breeding program is to create new cut flower Alstroemeria plants with desirable flower and plant qualities, attractive flower coloration and excellent postproduction longevity.

The new Alstroemeria plant originated from a cross-pollination made by the Inventor in Rijsenhout, The Netherlands in June, 2005, of two unnamed proprietary Alstroemeria hybrida selections, not patented. The new Alstroemeria plant was discovered and selected by the Inventor as a single flowering plant from within the progeny of the stated cross-pollination in a controlled greenhouse environment in Rijsenhout, The Netherlands in July, 2006.

Asexual reproduction of the new Alstroemeria plant by rhizome divisions in a controlled greenhouse environment in Rijsenhout, The Netherlands since September, 2006 has shown that the unique features of this new Alstroemeria plant are stable and reproduced true to type in successive generations.

### SUMMARY OF THE INVENTION

Plants of the new Alstroemeria have not been observed under all possible environmental conditions and cultural practices. The phenotype may vary somewhat with variations in environmental conditions such as temperature and light intensity without, however, any variance in genotype.

The following traits have been repeatedly observed and are determined to be the unique characteristics of ‘Zalsamida’. These characteristics in combination distinguish ‘Zalsamida’ as a new and distinct Alstroemeria plant:

1. Erect and strong flowering stems.

2. Vigorous growth habit.

3. Purple and white-colored flowers.

4. Excellent postproduction longevity.

Plants of the new Alstroemeria can be compared to plants of the parent selections. Plants of the new Alstroemeria differ from plants of the parent selections primarily in flower color as plants of the female parent selection have dark purple and yellow-colored flowers and plants of the male parent selection have light purple-colored flowers.

Plants of the new Alstroemeria can be compared to plants of Alstroemeria hybrida ‘Stabec’, disclosed in U.S. Plant Pat. No. 9,041. In side-by-side comparisons conducted in Rijsenhout, The Netherlands, plants of the new Alstroemeria differed primarily from plants of ‘Stabec’ in flower color as plants of ‘Stabec’ had light red to deep pink-colored flowers.

### BRIEF DESCRIPTION OF THE PHOTOGRAPH

The accompanying colored photograph illustrates the overall appearance of the new Alstroemeria plant showing the colors as true as it is reasonably possible to obtain in colored reproductions of this type. Colors in the photograph may differ slightly from the color values cited in the detailed botanical description which accurately describe the colors of the new Alstroemeria plant. The photograph comprises a close-up view of a typical flowering stem of ‘Zalsamida’.

### DETAILED BOTANICAL DESCRIPTION

The aforementioned photograph and following observations and measurements describe plants of the new Alstroemeria grown during the late summer and early autumn in ground beds in a glass-covered greenhouse in Rijsenhout, The Netherlands. During the production of the plants, day temperatures ranged from 15° C. to 25° C., night temperatures ranged from 10° C. to 15° C., soil temperatures averaged 15° C. and light levels averaged 5,000 lux. Plants were one year old when the photograph and description were taken. In the following description, color references are made to The Royal Horticultural Society Colour Chart, 2007 Edition, except where general terms of ordinary dictionary significance are used.
Botanical classification: Alstroemeria hybrida ‘Zalsamida’.
Parentage:
   Female, or seed, parent.—Unnamed proprietary Alstroemeria hybrida selection, not patented.
   Male, or pollen, parent.—Unnamed proprietary Alstroemeria hybrida, not patented.
Propagation:
   Type.—In vitro rhizogenesis.
   Time to produce a rooted young plant, summer.—About 40 days at 16° C. to 25° C.
   Time to produce a rooted young plant, winter.—About 60 days at 16° C. to 20° C.
   Root description.—Fibrous, fleshy, thick; color, close to 155D.
   Rooting habit.—Freely branching; medium density.
   Rhizomes.—Shape: Elongate; rounded. Length: About 10 cm to 30 cm. Diameter: About 3 mm to 10 mm. Texture: Smooth. Color: Close to 155D.
Plant description:
   Plant and growth habit.—Upright; freely branching, bushy appearance; vigorous growth habit.
   Plant height.—About 125 cm to 165 cm.
   Plant diameter (spread).—About 25 cm.
Flowering stem description:
   Aspect.—Erect.
   Length.—About 116 cm to 139 cm.
   Diameter.—About 8 mm to 10 mm.
   Internode length.—About 0.5 cm to 11.5 cm.
   Strength.—Strong.
   Texture.—Smooth, glabrous.
   Color.—Close to 137C; towards the base, tinged with close to N77A.
Foliage description:
   Appearance.—Leaves asymmetrical; sessile.
   Length.—About 17.3 cm to 20.5 cm.
   Width.—About 2.7 cm to 4.1 cm.
   Shape.—Elliptic.
   Apex.—Acute.
   Base.—Cuneate.
   Margin.—Entire.
   Texture, upper and lower surfaces.—Smooth, glabrous.
   Venation pattern.—Parallel.
   Color.—Developing and fully developed leaves, upper surface: Close to N137A; venation, close to N137A. Developing and fully developed leaves, lower surface: Close to N137C; venation, close to N137C.
Flower description:
   Flower type and habit.—Single cup-shaped flowers arranged in compound umbels; flowers face mostly outwardly; perianth segments separate; freely flowering habit, about 8 to 13 flowers developing per flowering stem.
   Natural flowering season.—Flowering continuous during the spring in The Netherlands; plants begin flowering about 80 to 90 days after planting.
   Fragrance.—Not detected.
   Flower longevity on the plant.—About two weeks; flowers not persistent.
   Flower longevity as a cut flower.—About 12 to 16 days; flowers not persistent.
   Flower buds (showing color).—Length: About 5 cm to 6 cm. Diameter: About 1.5 cm to 1.7 cm. Shape: Roughly ovoid. Color: Close to 59B.
   Umbel height.—About 14 cm to 19 cm.
   Umbel diameter.—About 20.5 cm to 24 cm.
   Flower diameter.—About 6 cm by 7 cm.
   Flower depth.—About 7 cm to 7.8 cm.
   Perianth.—Arrangement: Six arranged in two whorls, each whorl with two lateral and one median segments. Outer perianth, lateral segments: Length: About 6.2 cm to 6.5 cm. Width: About 3.6 cm to 4 cm. Shape: Obovate. Apex: Embedded pointed. Base: Attenuate. Margin: Entire; slightly undulate. Texture, upper and lower surfaces: Smooth, glabrous. Color, when opening and fully opened, upper surface: Close 77A. Color, when opening and fully opened, lower surface: Close to 77A. Outer perianth, median segment: Length: About 6.5 cm to 7 cm. Width: About 3.8 cm to 4.1 cm. Shape: Obovate. Apex: Embedded pointed. Base: Attenuate. Margin: Entire; slightly undulate. Texture, upper and lower surfaces: Smooth, glabrous. Color, when opening and fully opened, upper surface: Close to 77A. Color, when opening and fully opened, lower surface: Close to 77A. Inner perianth, lateral segments: Length: About 6.4 cm to 6.8 cm. Width: About 1.8 cm to 2.1 cm. Shape: Oblanceolate. Apex: Wishbone-shaped. Base: Attenuate. Margin: Entire; slightly undulate. Texture, upper and lower surfaces: Smooth, glabrous. Color, when opening and fully opened, upper surface: Close to 77A; towards the base, close to NN155D; at the base, close to 77D; stripes, close to 183A. Color, when opening and fully opened, lower surface: Close to 77A. Inner perianth, median segment: Length: About 5.1 cm to 5.5 cm. Width: About 1.8 cm to 2.1 cm. Shape: Oblanceolate. Apex: Wishbone-shaped. Base: Attenuate. Margin: Entire; slightly undulate. Texture, upper and lower surfaces: Smooth, glabrous. Color, when opening and fully opened, upper surface: Close to 77A. Color, when opening and fully opened, lower surface: Close to 77A.
   Pedicels.—Length: About 0.5 cm to 10 cm. Diameter: About 2 mm to 4 mm. Strength: Strong. Angle: About 20° from vertical. Texture: Smooth, glabrous. Color, upper and lower surfaces: Close to 137C tinged with close to 77A.
   Reproductive organs.—Stamens: Quantity per flower: Six. Anther shape: Elliptical. Anther length: About 9 mm to 11 mm. Anther color: Close to N187A. Pollen amount: Abundant. Pollen color: Close to N186A. Pistils: Quantity per flower: One. Style length: About 2 cm to 4 cm. Style color: Close to N77B. Stigma color: Close to N77B. Ovary color: Close to N77B.
   Seeds and fruits.—Seed and fruit development has not been observed on plants of the new Alstroemeria.
Disease & pest resistance: Plants of the new Alstroemeria have not been observed to be resistant to pathogens and pests common to Alstroemeria plants.
Temperature tolerance: Plants of the new Alstroemeria have been observed to tolerate temperatures from about 0° C. to about 40° C.

It is claimed:

1. A new and distinct Alstroemeria plant named ‘Zalsamida’ as illustrated and described.
